(19) United States
(12) Patent Application Publication
(10) Pub. No.: US 2019/0290494 A1
(43) Pub. Date: Sep. 26, 2019

(54) EAR PROTECTOR AND VISUAL DISPLAY AND METHOD OF USE

(71) Applicant: Arshad Mohabbat, St. Louis, MO (US)

(72) Inventor: Arshad Mohabbat, St. Louis, MO (US)

(21) Appl. No.: 16/362,359

(22) Filed: Mar. 22, 2019

Related U.S. Application Data
(60) Provisional application No. 62/646,427, filed on Mar. 22, 2018.

(51) Int. Cl. A61F 11/08

(52) U.S. Cl. CPC A61F 11/08; G10K 2210/1081

(57) ABSTRACT

A combination device for ear protection and visual display. The device includes an earplug configured to be removably seated with the user's ear canal, a visual display having an inner face and an outer face which serves as a platform for indicia, such as support for the user's favorite team. The device further includes a mount attached between the earplug and the display to connect the two. Methods of using the device are also provided.

### CROSS-REFERENCE TO RELATED APPLICATIONS

This Non-Provisional application claims priority to U.S. Provisional Application Ser. No. 62/646,427 filed Mar. 22, 2018, and which is incorporated herein by reference.

### STATEMENT REGARDING FEDERALLY SPONSORED RESEARCH

Not applicable.

### BACKGROUND

The present invention relates to ear protectors. More particularly, the present invention relates to a protective covering for the ear in conjunction with a visual display to display indicia.

Ear protection devices are used for a variety of different purposes. They can be used to protect against high decibel noises, such as music, sporting events, hunting, machinery, aircraft, automobiles, and the like. Other types of ear protection are used to protect against water or foreign objects from entering a person's ear canal. Ear protection can also be used to aid sleeping or lessen the effects or changes in air pressure, such as during flight.

Depending on the intended purposes, ear protection devices can be made from a variety of materials and in various shapes and designs. Disposable foam earplugs are commonly used for single use applications. For multiple use applications, reusable earplugs are generally made from plastic materials, such as silicone. For long-term uses, custom molded earplugs are made from high quality materials to fit the contours of a user's ear canal. Ear plugs can even incorporate electronics to provide special attenuation for musicians or other purposes.

Ear protection devices are commonly used at public venues for sporting events, concerts, racing events, and other events. At these types of events, it is common for fans to express their enthusiasm through all sorts of ways, such as, face painting, hair products, clothing, signs, jewelry, and other devices. These items can also be used by advertisers or sponsors to display various indicia.

What is needed is an ear protection device that allows fans or sponsors to show their enthusiasm, support, and indicia at various events.

### SUMMARY OF THE INVENTION

Applicant has provided a device which combines protection from noise, rain, cold or other reasons with an avenue to express their support or enthusiasm, or otherwise imparting a visual message using the same ear protector.

In one aspect, the invention is an ear protector and visual display in combination. The device includes an earplug configured to be removably seated with the user's ear canal. Also included is a display which has an inner face and an outer face which serves as a platform for indicia, such as support for the user's favorite team. The device further includes a mount attached between the earplug and the display to connect the two.

In a second aspect, an ear protection system configured to provide ear protection in combination with a visual display is provided. The system includes an earplug configured for removable seating with an ear canal, a display having an inner face and an outer face with indicia, and a mount attached between the earplug and the display, wherein the indicia are selected from those expressing the user's enthusiasm, support or providing a message of choice.

In yet another aspect, the invention provides a method for providing for ear protection and visual display in combination. The method includes the steps of obtaining a device which includes both an ear protector and visual display. The device includes an earplug which is configured to be removably seated with the user's ear canal. Also included in the device is a display which has an inner face and an outer face which serves as a platform for indicia, such as support for the user's favorite team. The device also includes a mount attached between the earplug and the display to connect the two. The method further includes placing the ear protector on the user's ear so that it seats with the ear canal, and so that the visual display is visible.

### DESCRIPTION OF THE DRAWINGS

In the accompanying drawings which form part of the specification:

FIG. 1 is a perspective view of an ear protector device, constructed in accordance with the present disclosure;

FIG. 2 is a side view of the ear protector device, constructed in accordance with the present disclosure;

FIG. 3 is a rear view of the ear protector device, constructed in accordance with the present disclosure;

FIG. 4 is a front view of the ear protector device, constructed in accordance with the present disclosure;

FIG. 5 is a perspective view of an ear bud of the ear protector device, constructed in accordance with the present disclosure;

FIG. 6 is a perspective view of an ear bud mount of the ear protector device, constructed in accordance with the present disclosure; and

FIG. 7 is a perspective view of alternative embodiment of an ear protector device, constructed in accordance with the present disclosure.

Corresponding reference numerals indicate corresponding parts throughout the several figures of the drawings.

### DETAILED DESCRIPTION

The following detailed description illustrates the claimed invention by way of example and not by way of limitation. The description clearly enables one skilled in the art to make and use the disclosure, describes several embodiments, adaptations, variations, alternatives, and uses of the disclosure, including what is presently believed to be the best mode of carrying out the claimed invention. Additionally, it is to be understood that the disclosure is not limited in its application to the details of construction and the arrangements of components set forth in the following description or illustrated in the drawings. The disclosure is capable of other embodiments and of being practiced or being carried out in various ways. Also, it is to be understood that the phraseology and terminology used herein is for the purpose of description and should not be regarded as limiting.

As shown in FIGS. 1-7, an embodiment constructed in accordance with the present disclosure, generally referred to as an ear protector device 10, includes an earplug 12 connected to a display 14 with a mount 16. The display 14 includes an outer face for displaying indicia 22.

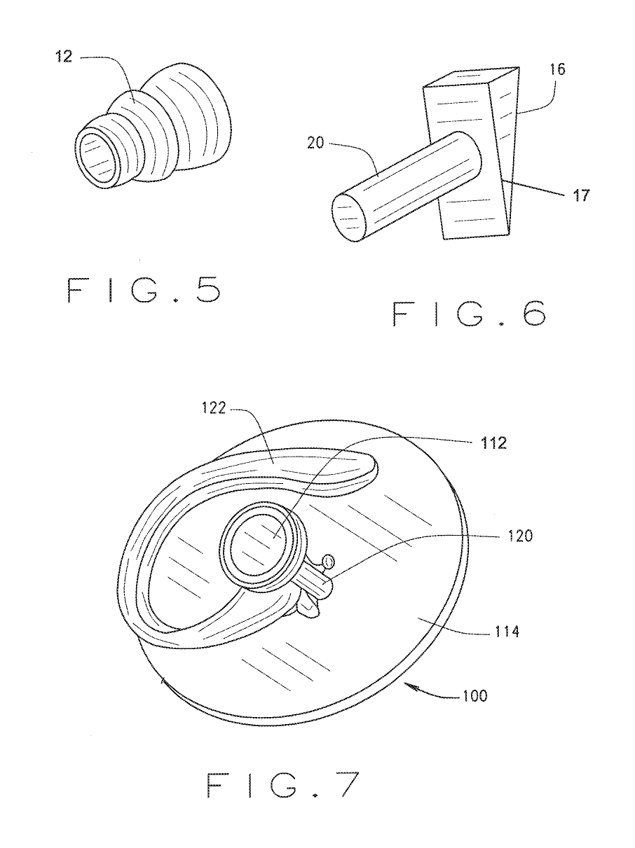

As shown in FIGS. 1 and 5, the earplug 12 is a generally conical shaped member shaped and sized for insertion and engagement with a user's ear canal (not shown). Those skilled in the art will recognize that the earplug can be any suitable shape, size, or type, including but not limited to, flanged, hi-fi, electronic noise cancellation, and the like. The earplug 12 is preferably made from a plastic material, such as silicone. The earplug is preferably made from a soft plastic material, such as silicone. However, those skilled in the art will recognize that the earplug can be made from any suitable material, including, but not limited to, foam, memory foam, polyvinyl chloride, polyurethane, metal, wax, or combination thereof.

As shown in FIGS. 1-3, the display 14 is a generally circular disc having an outer face and an inner face. Indicia 22 is displayed on the outer face. In the present embodiment, the indicia 22 is molded into the outer face. However, the indicia 22 can be placed on the outer surface using any suitable method, including, but not limited to, adhesive, painting, printing, fasteners, welding, and the like. In alternate embodiments, indicia can also be displayed on the inner face. Although FIGS. 1-6 disclose a display as a generally circular disc, and suitable shape and size can be used, including, but not limited to, rectilinear, curvilinear, or other shapes.

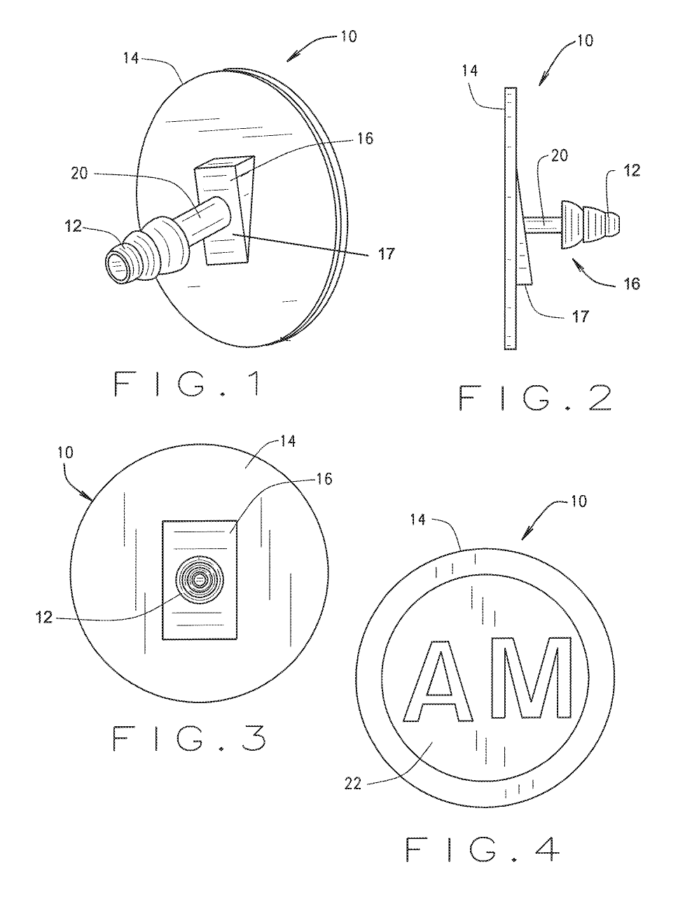

As shown in FIG. 2, the mount 16 is a generally triangular or angled wedge 17 mounted to about the center of the display with a post 20 extending outwardly to attach to the earplug 12. The earplug 12 engages the post 20 using any suitable method, including, but not limited to, friction, adhesive, painting, printing, fasteners, welding, and the like. The wedge 17 is configured so that the post 20 is positioned at about a 15-30° angle relative to the display 14. In an alternative embodiment, the post 20 can be directly attached to the display 14.

In operation, the user inserts and seats respective earplugs 12 of a pair of ear protection devices 10 into his or her respective ear canals. In this position, the displays 14 are positioned on each side of the user's head adjacent the ears.

FIG. 7 depicts an alternate embodiment of an ear protector device 100, which includes an earplug 112 connected to a display 114 with a mount. The display 14 include an outer face for displaying indicia 22.

The earplug 112 is a generally disc shaped member shaped and sized for insertion and engagement with a user's ear canal. Those skilled in the art will recognize that the earplug can be any suitable shape, size, or type, including but not limited to, flanged, hi-fi, electronic noise cancellation, and the like. The earplug 112 is preferably made from a plastic material, such as silicone. The earplug 112 is preferably made from a soft plastic material, such as silicone. However, those skilled in the art will recognize that the earplug can be made from any suitable material, including, but not limited to, foam, memory foam, polyvinyl chloride, polyurethane, metal, wax, or combination thereof. An ear hook 124 extends from the earplug 112. The ear hook 122 is generally hook or C-shaped to engage around the outer portion of the user's ear for support of the device 100.

As shown in FIGS. 4 and 7, the display 14 is a generally circular disc having an outer face and an inner face. Indicia 22 is displayed on the outer face. In the present embodiment, the indicia 22 is molded into the outer face. However, the indicia 22 can be placed on the outer surface using any suitable method, including, but not limited to, adhesive, painting, printing, fasteners, welding, and the like. In alternate embodiments, indicia can also be displayed on the inner face. Although FIGS. 4 and 7 disclose a display as a generally circular disc, and suitable shape and size can be used, including, but not limited to, rectilinear, curvilinear, or other shapes.

As shown in FIG. 7, the mount is a post 120 extending outwardly from the display 114 to attach to the earplug 112. The earplug 112 engages the post 120 using any suitable method, including, but not limited to, friction, adhesive, painting, printing, fasteners, welding, and the like.

Changes can be made in the above constructions without departing from the scope of the disclosure, it is intended that all matter contained in the above description or shown in the accompanying drawings shall be interpreted as illustrative and not in a limiting sense. For example, in alternative embodiments, the display can also include lighting arrangements, such as LED, Electroluminescent wire, or other suitable lighting. In addition the lighting arrangement can include a suitable power source, such as battery or solar. In another alternative embodiment, the ear protection device can include other electronic devices, including but not limited to, audio devices, speakers, wireless devices, such as Bluetooth or Wi-Fi, and the like.

What is claimed is:

1. An ear protector device, comprising:
an earplug configured for removable seating with an ear canal;
a display having an inner face and an outer face with indicia; and
a mount attached between the earplug and the display.

2. The ear protector device of claim 1 wherein the earplug is selected from the group consisting of one or more of a flanged, hi-fi, electronic and noise-cancellation earplugs.

3. The ear protector device of claim 1 wherein the inner face of the display further comprises indicia.

4. The ear protector device of claim 1 wherein the device further comprises a post between the mount and the display configured to extend the device outwardly from the mount.

5. An ear protection system configured to provide ear protection in combination with a visual display, the system comprising:
an earplug configured for removable seating with an ear canal;
a display having an inner face and an outer face with indicia; and
a mount attached between the earplug and the display,
wherein the indicia are selected from those expressing the user's enthusiasm, support or providing a message of choice.

6. The ear protection system of claim 5 wherein the earplug is configured to provide for enhanced noise-cancellation through the ear canal.

7. The ear protection system of claim 5 wherein the system further comprises a post between the mount and the display configured to extend outwardly from the mount to the display.

8. A method for providing for ear protection and visual display in combination, the method comprising:
the step of obtaining a device which comprises an earplug configured for removable seating with an ear canal; a display comprising an inner face and an outer face adapted to serve as a platform for indicia, and a mount attached between the earplug and the display to connect the earplug and the display; and
the step of placing the ear protection and visual display device on a user's ear so that it seats with the ear canal, and so that the visual display is visible.

9. The method of claim 8 further comprising the step of attaching indicia to the display prior to placing the device on the user's ear.

10. The method of claim 8 further comprising the step of making the device prior to obtaining and placing the device on the user's ear.
